(19) United States
(12) Patent Application Publication
(10) Pub. No.: US 2018/0104513 A1
(43) Pub. Date: Apr. 19, 2018

(54) Method and apparatus for an ion beam accelerator and beam delivery system integrated on a rotating gantry

(71) Applicant: Alan Sliski, Lincoln, MA (US)

(72) Inventor: Alan Sliski, Lincoln, MA (US)

(21) Appl. No.: 15/293,648

(22) Filed: Oct. 14, 2016

(51) Int. Cl. A61N 5/10

(52) U.S. Cl. CPC A61N 5/1081; A61N 5/1043; A61N 5/1071; A61N 2005/1087; A61N 5/1049; A61N 5/1067; A61N 5/103

(57) ABSTRACT

An integrated system for radiation therapy that includes a synchrotron ion accelerator beam delivery system, and imaging system on the same compact rotating gantry is disclosed. An ion accelerator, accelerating charged particles, is mounted on an annular gantry that rotates about the patient to be treated. The charged particles may be protons or other heavier ions such as helium or carbon. The beam delivery system, which includes bending, focusing, and scanning magnets and other equipment for accurately delivering the ion beam to the patient, is integrated on the same rotating structure. An imaging system which in one embodiment is, but not limited to, a cone beam CT system. The accelerator and imaging system may be configured in a coplanar or non-coplanar arrangement with the rotation plane of the gantry and with the beam delivery system to allow for optimization of the size or functionality of the system.

### FIELD OF THE INVENTION

The invention discloses devices and methods employing a particle accelerator and associated system components that are used for ion based radiation therapy. Protons are the most common ion used for radiation therapy of cancer. Carbon and helium ions are also presently used. An integrated imaging system that uses x-rays or one of several other methods including prompt gamma emission from the beam as it interacts with the anatomy of the patient would optionally be included in the integrated system along with the control system hardware and software, beam diagnostic equipment, patient positioning and safety systems.

### BACKGROUND OF THE INVENTION

Ion accelerators have been proposed to treat cancer with radiation since the 1940's. The advantage over high energy X-rays is that the energy deposition peaks at the “Bragg peak” and stops at a predetermined and programmable distance within the patient. Historically, high energy physics research laboratories first pioneered the use of protons for treating cancer, among the earliest being the Harvard cyclotron. Accelerators are typically fixed, heavy machines. The proton beam is delivered through an evacuated tube, guided by magnets to a rotating gantry that rotates around a moveable patient couch. This gantry contains bending magnets and a beam delivery system to position and condition the beam for treatment. By rotating the gantry and choosing the patient position and orientation with the couch, the proton beam can be brought to the desired treatment volume within the patient. An imaging system, often an X-ray based cone beam CT system aids in aligning the patient to the machine. The accelerator and gantry are often separate systems. One commercial system, from Mevion Corporation, mounts a superconducting synchrocyclotron on a 180 degree gantry for patient treatment. This disclosure relates to mounting a competing type of accelerator, a synchrotron, on an annular gantry that can rotate through 360 degrees around the patient, along with the related support and imaging systems. The synchrotron is comprised of bending magnets that are arranged in a roughly circular shape, comprised of circular segments that are bending magnets, with interconnecting straight sections of evacuated tubes that contain the RF accelerator subsystem, focusing magnets, beam diagnostics and related systems. A beam injector, often comprising a radio frequency quadrupole, accelerates the ions from an ion source to a moderate energy, often about 5-7 MeV, where the synchrotron can increase the energy to the desired energy for treatment, up to about 350 MeV. The synchrotron is usually coupled to a beam transport system that selectively brings the beam to one or more treatment gantries. The gantry magnets bend the beam into a trajectory radially inward toward the patient on the couch, and also hold the beam delivery system which may employ scanning magnets or scattering systems to modulate the position of the beam in 3 dimensions within the patient, based on prior imaging studies and treatment planning software.

### SUMMARY OF THE INVENTION

The integrated accelerator/gantry system includes the following elements
1. An ion source that generates the low energy ions that are coupled to the injector
2. An injector that accelerates the ions to moderate energy suitable for coupling into the synchrotron accelerator.
3. A synchrotron that accelerates and shapes the ion beam to the desired energy and geometry.
4. A beam extraction subsystem that extracts the stored high energy ions and delivers them to the treatment nozzle.
5. A treatment nozzle that accepts the ion beam at the desired energy and deflects the ion beam to the desired areas within the patient. The nozzle includes scanning magnets or a scattering system to generate the beam energy and profile within the patient. The nozzle also includes diagnostic instrumentation to assure that the beam is of the correct energy and position. The nozzle may optionally include imaging system components for X-ray or ion beam imaging studies of the patient to plan and confirm the treatment delivery.
6. A structure that supports the accelerator and related systems in an accurate and repeatable manner. The gantry structure also optionally supports the associated equipment such as the RF system and magnet power supplies. The gantry rotates on a programmable drive system that positions the gantry in at least one rotational axis. The gantry may support the accelerator within it in a non-coplanar orientation, where the beam accelerator plane is different than the rotational plane. This rotating structure may optionally include imaging means for determining the position of the anatomy of the patient, and the ion beam within the patient.
7. A support structure for the gantry, couch and cosmetic covers. This support structure may include the structural shielding for the system, typically made of thick concrete. The gantry structure may be separately mounted within the structural shielding, or use the structural shielding as an integral part of the gantry support substructure.

### BRIEF DESCRIPTION OF THE DRAWINGS

FIGS. 1a-c shows one illustrative coplanar configuration of the accelerator and gantry with three views. Several functional elements that may be used in a commercial implementation such as a second gantry ring are omitted for clarity.

### DETAILED DESCRIPTION OF INVENTION

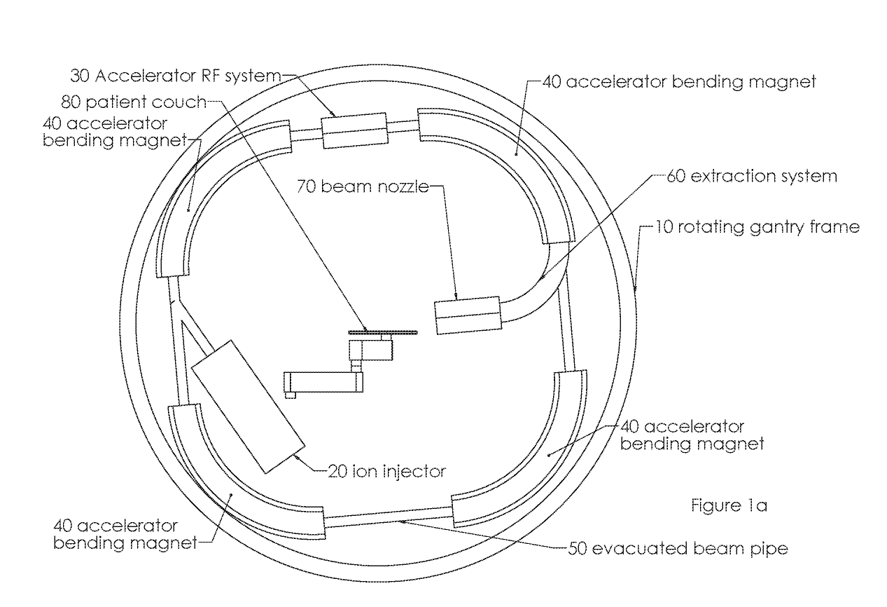

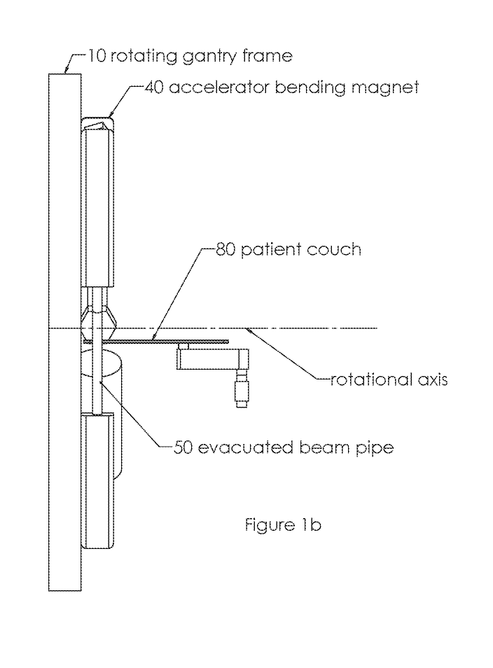

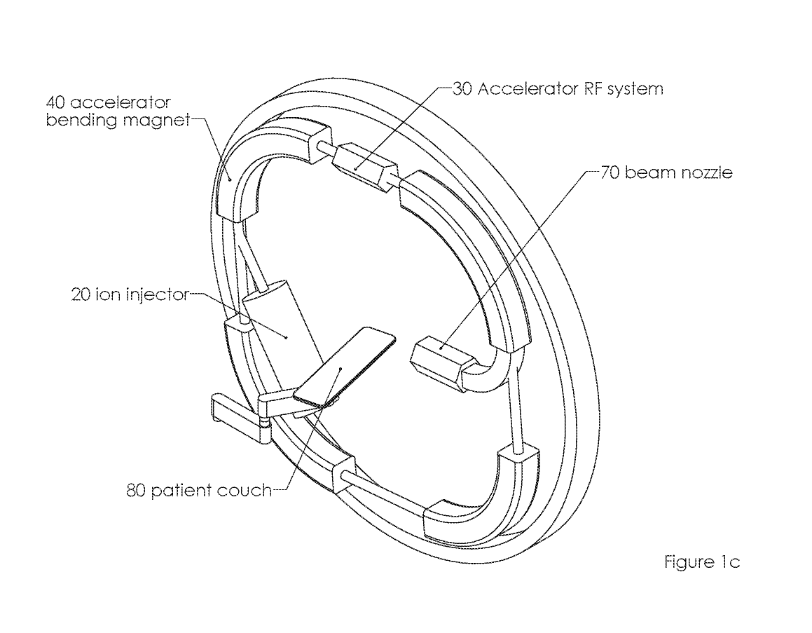

FIG. 1 illustrates one possible configuration of the accelerator mounted within the rotating gantry structure where the plane of acceleration is coplanar with the rotational plane of the gantry. FIG. 1a is a view perpendicular to the axis of rotation. Rotating gantry frame 10 is a structural ring that is supported by bearings or rollers, not shown, and rotated by a drive system, not shown, which can rotate the gantry to a predetermined angle and turn it at a predetermined speed when commanded. Gantry frame 10 supports all of the elements of the accelerator in a rigid alignment. This support structure is omitted for clarity. Ion injector 20 is shown in one possible orientation that will inject moderate energy ions into the synchrotron accelerator at the appropriate energy and timing for coupling the beam into the synchrotron accelerator ring. The accelerator RF system 30 increases the ion beam energy each orbit of the ions around the accelerator, up to the desired maximum energy. The bending magnets 40, bend the ions so they continue to stable orbit the accelerator. The magnetic field is ramped during acceleration to match the energy of the increasing ions, including the relativistic mass increase as the ions reach higher energy. A control system, not shown, controls the injector 20, RF system 30, magnets 40, extraction system 60, the gantry system, couch 80, and beam nozzle 70 to deliver a useful beam to the patient at the predetermined position and energy sequence.

The evacuated beam pipe 50 encloses the beam path and maintains high vacuum, with vacuum pumps so that the ions can travel without colliding with gas molecules. The beam pipe includes a continuous ring through the magnets 40, RF section 30, extraction system 60, and injector 20. Focusing magnets may be interspersed in the beam path, or the focusing function may optionally be included in the bending magnets. Beam diagnostic instruments are also included in the system to measure the position and energy of the beam as it is accelerated , stored and dumped to the beam delivery subsystem and nozzle.

The extraction system 60 includes electrodes and/or magnets within the accelerator beam pipe to strip off protons that have been accelerated to the predetermined energy and direct them substantially radially inward toward the patient.

The beam nozzle 70 contains instrumentation for measuring one or more of the beam energy, current, and position. The beam nozzle 70 also contains means for creating a large beam effective area, matched to the target volume shape in three dimensions. This can be accomplished with a scattering system and apertures, or with a pencil beam scanning system, which uses scanning magnets to scan the beam and associated Bragg peak within the target volume, to deliver the predetermined dose to the predetermined treatment volume. The nozzle 70 or separate annular structure also optionally contains parts of an imaging system such as an X-ray tube used to generate radiographs of the patient prior to and during treatment. The ion beam can also be used as a source of radiation to be used with other imaging detectors to visualize the prompt gammas generated by the interaction of the ion beam with the patient tissue. This helps localize the beam within the patient, and can be used for real time feedback of the beam position, improving the dose conformity.

The patient couch 80 supports the patient inside the rotating gantry in the predetermined position and orientation for the treatment. A two arm robotic couch is shown, which can have motion in six degrees of freedom. Several configurations of robotic patient couch are in use commercially, and several configurations can be adapted to use in this system. A cosmetic structure which provides a floor for the patient and therapist to walk on is not shown, but will be included in a finished installation. Walls, lighting, air conditioning etc. are also included in a finished system but not shown for clarity.

FIG. 1b shows a view parallel to the rotation axis of the gantry. FIG. 1c shows an isometric view of the same illustrative embodiment.

The optional integrated imaging system using prompt gamma emission from the patient can be used to adjust the couch position and/or the beam energy and position to correct for tissue inhomogeneity such as lung and bone, where the treatment planning images obtained using X-rays may be less precisely predictive of the ion beam range than desired, or the registration between the planning images and the target volume position has varied due to organ motion, for example.

The invention claimed is:

1. An apparatus comprising an integrated ion accelerator treatment system and rotating gantry for radiation therapy of a patient with cancer.

2. An apparatus of claim 1 where the accelerator beam plane and the rotational plane of the gantry are parallel.

3. An apparatus of claim 1 where the accelerator beam plane and the rotational plane of the gantry are tilted with respect to each other.

4. An apparatus of claim 1 that includes an imaging system.

5. An apparatus of claim 2 that includes an imaging system.

6. An apparatus of claim 3 that includes an imaging system.

7. A method for controlling the delivery of ion therapy using the information gathered by the imaging system during the treatment.

8. An apparatus of claim 1 where a patient imaging system is integrated with the rotating gantry

9. An apparatus of claim 2 where a patient imaging system is integrated with the rotating gantry

10. An apparatus of claim 3 where a patient imaging system is integrated with the rotating gantry

11. An apparatus of claim 8 where the integrated patient imaging system is mounted to, but rotates independently from the main gantry on a coaxial, coplanar or non-coplanar subgantry.

12. An apparatus of claim 9 where the integrated patient imaging system is mounted to, but rotates independently from the main gantry on a coaxial, coplanar or non-coplanar subgantry.

13. An apparatus of claim 10 where the integrated patient imaging system is mounted to, but rotates independently from the main gantry on a coaxial, coplanar or non-coplanar subgantry.
